(19) United States
(12) Patent Application Publication
(10) Pub. No.: US 2026/0221224 A1
(43) Pub. Date: Jul. 30, 2026

(54) SPATIALLY INFORMED GENOMIC SEQUENCING

(71) Applicant: CANON MEDICAL SYSTEMS CORPORATION, Otawara-shi (JP)

(72) Inventors: Simon FISHER, Edinburgh (GB); Jacob BRADLEY, Edinburgh (GB); Russell HUNG, Edinburgh (GB)

(73) Assignee: CANON MEDICAL SYSTEMS CORPORATION, Otawara-shi (JP)

(21) Appl. No.: 19/037,010

(22) Filed: Jan. 24, 2025

(51) Int. Cl. G16B 20/20; C12Q 1/6806; G16H 20/10; G16H 20/40; G16H 50/20

(52) U.S. Cl. CPC G16B 20/20; C12Q 1/6806; G16H 20/10; G16H 20/40; G16H 50/20

(57) ABSTRACT

A method for analysing a tissue sample comprising receiving spatial transcriptomics data based upon the tissue sample, the spatial transcriptomics data comprising, for a plurality of spatial locations on the sample, expression values of a plurality of genes associated with one or more cells located at the plurality of spatial locations; for at least a subset of the spatial locations, classifying the corresponding one or more cells as cancerous or non-cancerous based upon the associated expression values; and determining a boundary of a cancerous region of the tissue sample based upon the spatial locations classified as corresponding to one or more cancerous cells.

### FIELD

The present disclosure relates to systems and methods to analyse a tissue sample using spatial transcriptomics, and in particular to monitor tissue resection.

### BACKGROUND

Surgical resection is a process by which a tissue or structure is removed from a patient's body. Surgical resection can be performed on cancer patients to remove a tumour with the aim of removing the entire tumour. In some cases, the resection may be incomplete, meaning that some amount of cancerous tissue is left at the site of the surgery. This is associated with increased likelihood of further disease progression.

After surgical resection of a tumour, it is important that clinicians are able detect whether the resection was complete. Existing methods for identifying incomplete resection rely on a small number of visual and biological markers, and do not provide any means of understanding the specific characteristics of the residual diseased tissue. Neither do these existing approaches provide clinicians with any specific biological information about the tumour tissue remaining in the body.

Incomplete resection can be identified by the detection of “positive margins” on the resected tissue using immunohistochemistry or histology approaches. Positive margins are regions of the resected tissue indicating that tumour cells are present. These margins may be located on the boundary of the resected region. However, immunohistochemistry techniques are low throughput, since only one stain is typically used to analyse a tissue sample. An inappropriate choice of stain can lead to inaccurate results, such as a false-negative, or, less commonly, a false-positive. Multiplex markers can be more informative than a single stain and their use lowers the chance of inaccurate result. However, this approach fails to utilise the full biological information available from resected samples.

Omics is the study of quantities of biomolecules within a single cell or a population of cells. Transcriptomics is the study of the quantities of RNA molecules. When a transcriptomics analysis focusses on only those RNA molecules, which are encoded by genes, the transcriptomics analysis may be termed gene expression analysis. Spatial transcriptomics allows for spatially-resolved, high-throughput profiling of single, or a small number of cells of a tissue sample. Spatial transcriptomics data comprises the spatial locations of one or more cells in the tissue sample and corresponding gene expression data. The rich datasets obtained from spatial transcriptomics allows for the subsequent identification of cell types and states for the plurality of spatial locations of a tissue sample.

### BRIEF DESCRIPTION OF FIGURES

Arrangements of the present disclosure will be understood and appreciated more fully from the following detailed description, made by way of example only and taken in conjunction with drawings in which:

FIG. 1 illustrates a computing apparatus according to an embodiment;

FIG. 2 illustrates a resected tissue sample analysed by an immunohistochemistry method according to the prior art;

FIGS. 3A and 3B illustrate a method to analyse a resected tissue sample according to an embodiment;

FIG. 4 illustrates a method to classify a spatial location of the resected tissue sample using gene set enrichment analysis.

FIG. 5 illustrates a method to classify a spatial location of the resected tissue sample using machine learning.

FIG. 6 illustrates a method to make clinical decisions based on mutational profile data.

### DESCRIPTION

Certain embodiments provide a method for analyzing a tissue sample comprising receiving spatial transcriptomics data based upon the tissue sample, the spatial transcriptomics data comprising, for a plurality of spatial locations on the sample, expression values of a plurality of genomic nucleic acids associated with one or more cells located at the plurality of spatial locations; for at least a subset of the spatial locations, classifying the corresponding one or more cells as cancerous or non-cancerous based upon the associated expression values; and determining a boundary of a cancerous region of the tissue sample based upon the spatial locations classified as corresponding to one or more cancerous cells.

Certain embodiments provide a processing apparatus comprising a processing circuitry configured to: receive spatial transcriptomics data based upon the tissue sample, the spatial transcriptomics data comprising, for a plurality of spatial locations on the sample, expression values of a plurality of genomic nucleic acids corresponding to one or more cells located at the plurality of spatial locations; for at least a subset of the spatial locations, classify the corresponding one or more cells as cancerous or non-cancerous based upon the associated expression values; and determine a boundary of a cancerous region of the tissue sample based upon the spatial locations classified as corresponding to one or more cancerous cells.

Certain embodiments provide a non-transitory storage medium storing therein a program that causes a computer to execute the steps of: receive spatial transcriptomics data based upon the tissue sample, the spatial transcriptomics data comprising, a plurality of spatial locations on the sample, expression values of a plurality of genomic nucleic acids associated with one or more cells located at the plurality of spatial locations; for at least a subset of the spatial locations, classify the corresponding one or more cells as cancerous or non-cancerous based upon the associated expression values; and determine a boundary of a cancerous region of the tissue sample based upon the spatial locations classified as corresponding to one or more cancerous cells.

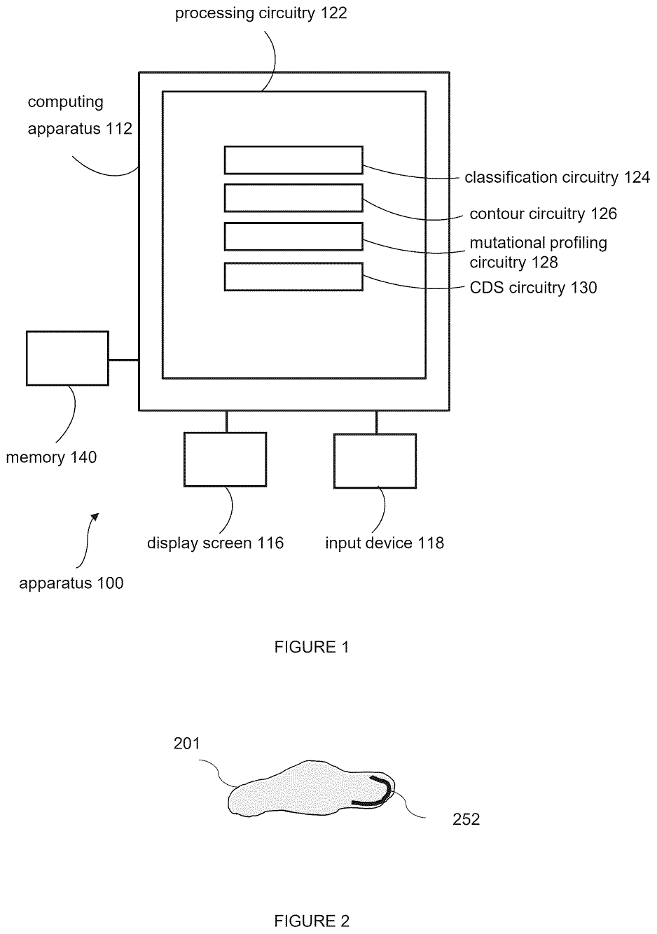

An apparatus 100 according to an embodiment is illustrated schematically in FIG. 1. The apparatus 100 may also be referred to as a medical information processing apparatus. The apparatus 100 is configured to process spatial transcriptomics data and gene sequencing data. The apparatus 100 is further configured to display analysis results based on the processed spatial transcriptomics data and processed gene sequencing data. In other embodiments, the apparatus 100 may be configured to process any appropriate data, which may comprise non-omics data, such as any unordered data.

The apparatus 100 comprises a computing apparatus 112, which in this case is a personal computer (PC) or workstation. The computing apparatus 112 is connected to a display screen 116 or other display device, and an input device or devices 118, such as a computer keyboard and mouse. The computing apparatus 112 receives data from memory 140, which may also be referred to as a data store or storage. In alternative embodiments, computing apparatus 112 receives data from one or more further data stores (not shown) instead of or in addition to memory 140. For example, the computing apparatus 112 may receive data from one or more remote data stores (not shown), which may comprise cloud-based storage.

The memory 140 stores spatial transcriptomics data, which quantifies the amounts of transcripts at a plurality of spatial locations of one or more tissue samples for one or more subjects. The memory 140 further stores gene sequencing data obtained from one or more tissue samples for one or more subjects. In other embodiments, the spatial transcriptomics data and/or gene sequencing data may be stored in another suitable memory, for example in another apparatus or in a cloud-based memory.

Computing apparatus 112 comprises a processing apparatus 122 for processing data. The processing apparatus 122 comprises a central processing unit (CPU) and Graphical Processing Unit (GPU). The processing apparatus 122 provides a processing resource for automatically or semi-automatically processing the transcriptomics data 100.

The processing apparatus 122 includes a classification circuitry 124 configured to process the spatial transcriptomics data and determine spatial locations of the tissue sample as healthy or cancerous, a contour circuitry 126 configured to identify borders of cancerous cells on the tissue sample, a mutational profiling circuitry 128 configured to analyse gene expression data and determine the mutational profile of cancerous cells of the tissue sample, and optionally, a clinical decision support (CDS) circuitry 130 configured to provide clinical support based on the mutational profile.

In the present embodiment, the circuitries 124, 126, 128 and 130 are each implemented in the CPU and/or GPU by means of a computer program having computer-readable instructions that are executable to perform the method of the embodiment. In other embodiments, the circuitries may be implemented as one or more ASICs (application specific integrated circuits) or FPGAs (field programmable gate arrays).

The computing apparatus 112 also includes a hard drive and other components of a PC including RAM, ROM, a data bus, an operating system including various device drivers, and hardware devices including a graphics card. Such components are not shown in FIG. 1 for clarity.

Turning to FIG. 2, the analysis of a resected sample using immunohistochemistry will now be described. A tissue sample 201 is obtained from a larger tissue sample. The tissue sample 201 may be a slice of the larger tissue sample, which comprises a tumour removed from a patient during surgery. A stain comprising antibodies, which bind to a target tumour antigen is applied to the sample 201. If the tumour antigen is present, the staining will produce one or more positive margins 252 on the tissue sample 201, which indicate regions where tumour cells are present. If, based on visual inspection, the positive margin is determined to be at the edge of the resection sample 201, the resection may be considered to be incomplete.

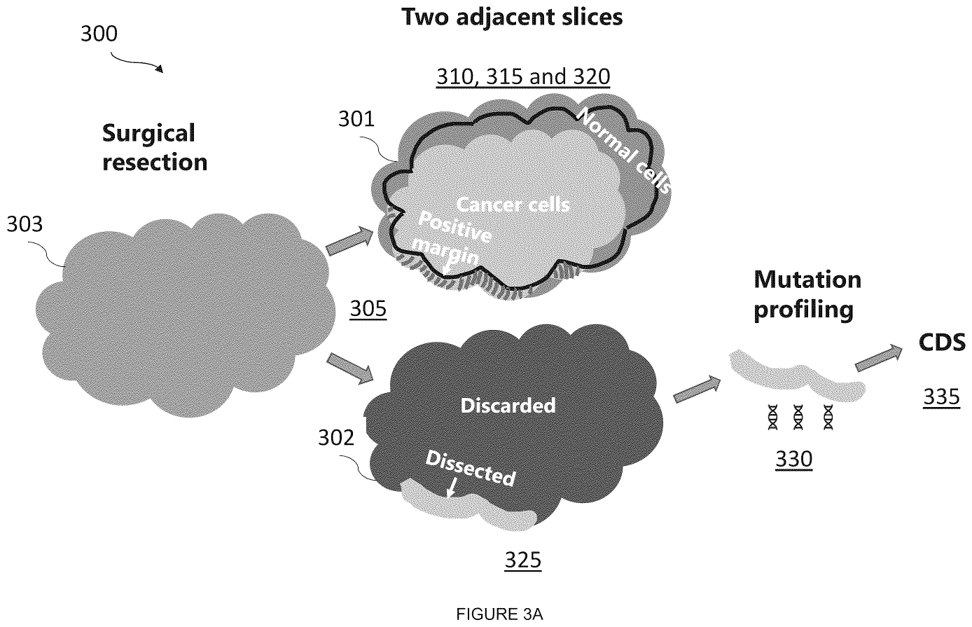

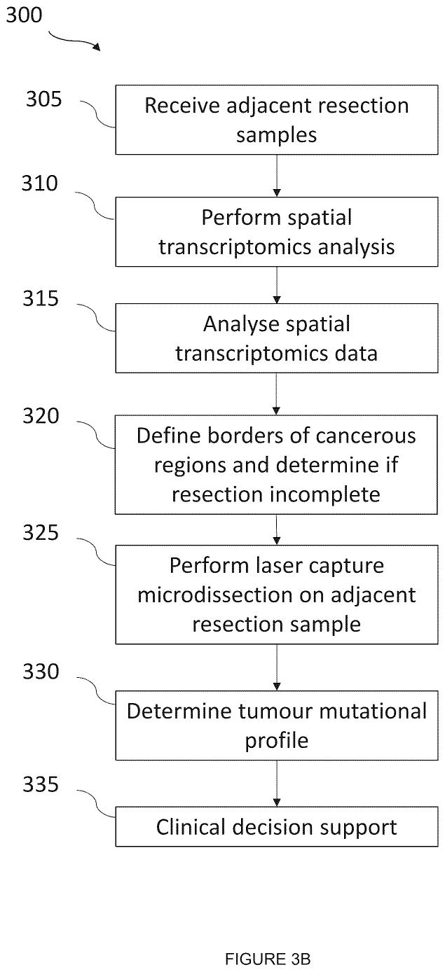

Turning to FIGS. 3A and 3B, a method 300 for analyzing a resection sample, which leverages spatial transcriptomics will now be described. At 305, two respective tissue resection samples 301 and 302 are obtained. Resection samples 301 and 302 are adjacent slices from a larger tissue resection sample 303 obtained from a patient from tumour resection surgery. At 310, spatial transcriptomics is performed on the resection sample 301 to generate spatial transcriptomics data. At 315, the spatial transcriptomics data is received by the computer apparatus 112 and plurality of spatial locations of the resection sample are classified as corresponding to either cancerous or healthy tissue. At 320, the borders of cancerous regions are defined on the tissue sample. At 325, laser capture microdissection is performed on a second resection sample 302 at one or more regions defined by the borders of cancerous regions and the obtained subsamples are then transcriptomally profiled, typically by RNA sequencing. At 330, the profiling, or sequencing data is received by the computing apparatus 112 and analysed to determine a tumour mutational profile. At 335, clinical support decisions are made based on the tumour mutational profile by a clinician and/or the computing apparatus 112.

Considering now the method 300 in further detail, at 305, the resection samples 301, 302 may be a thin slices between 5 to 10 μm. The way in which the resection samples 301, 302 are prepared may be dependent upon the type of spatial transcriptomics technique applied to resection sample 301. For example, if a spatial transcriptomics technique for fresh frozen tissue is to be used, the resection samples 301, 302 may be obtained by freezing the larger tissue sample 303 and cryosectioning the larger tissue sample 303 into slices. Alternatively, if a spatial transcriptomics technique for formalin-fixed paraffin embedded (FFPE) tissue is to be used, the resection samples 301, 302 may be obtained by first fixing, dehydrating, clearing and paraffin embedding the larger tissue sample 303 to result in a FFPE tissue block, and then cutting a slice of the FFPE tissue block.

At 310, a spatial transcriptomics technique is applied to the resection sample 301 to result in the generation of spatial transcriptomics data. In one example, the spatial transcriptomics technique is a 10xGenomics Visium protocol for fresh frozen tissue or FFPE tissue as described in Visium Spatial Protocols—Tissue Preparation Guide, (CG000240, 10xGenomics), which comprises the following steps:
i) capturing RNA contained in cells of the tissue sample on a test slide comprising capture areas using barcoded mRNA-binding oligonucleotides,
ii) imaging the test slide,
iii) preparing a sequencing library,
iv) performing next generation sequencing using the library, and
v) performing bioinformatics analysis on the sequencing and imaging data to generate spatial transcriptomics data 340.

It can be understood that any type of suitable spatial transcriptomics method can be applied at 310. For example, the CosMX Spatial Multiomics Single-Cell Imaging Platform (Nanostring) may be used to perform spatial transcriptomics on a tissue sample to generate spatial transcriptomics data 340. An example of the use of the CosMX Spatial Multiomics Single-Cell Imaging Platform is described in Williams, Claire, et al. “Spatial insights into tumor immune evasion illuminated with 1000-plex RNA profiling with CosMx Spatial Molecular Imager.” Cancer Res 83.6765 (2023): 10-1158.

The resultant spatial transcriptomics data 340 comprises, for the plurality of spatial locations on the sample, corresponding quantities of a plurality of transcripts, i.e. gene expression levels, associated with one or more cells located at the plurality of spatial locations. Depending on the type of spatial transcriptomics technique used, each of the plurality of spatial locations may correspond to either a single cell (which may be, for instance, if the CosMX Spatial Multiomics Single-Cell Imaging Platform is used), or to one or more cells (which may be, for instance, if a 10X Genomics platform is used).

The plurality of spatial locations comprises a set of 2D co-ordinates corresponding to a location on the resection sample 301. Each spatial location may comprise an x and y position.

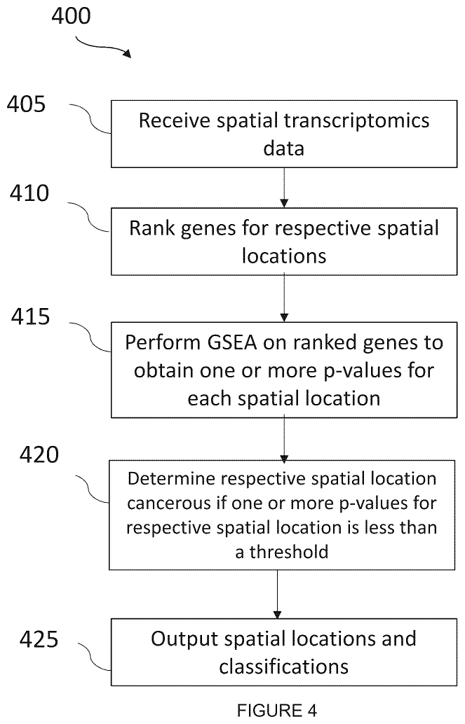

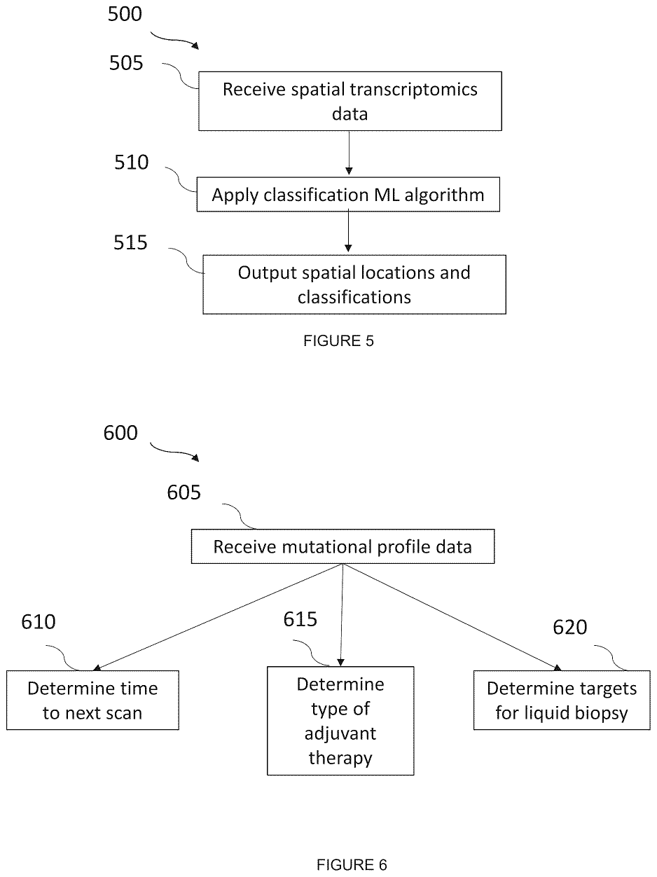

At 315, the spatial transcriptomics data 340 is received by the classification circuitry 124 and analysed to classify at least a subset of the spatial locations as corresponding to healthy or cancerous tissue. In one embodiment, the classification is performed according to a gene set enrichment analysis approach 400, which is explained in further detail with reference to FIG. 4. In another embdiment, a machine-learning algorithm is used to perform the classification with reference to FIG. 5. It will be understood that a variety of bioinformatics methods may be suitable for classifying the spatial locations as corresponding to healthy or cancerous tissue. For example, other methods may look at the expression of cancer-specific gene modules relative to expression levels in those modules for reference or healthy tissue.

The output from this step is a classification for each of the spatial locations that were analysed.

At 320, the spatial locations and corresponding classifications are received by the contour circuitry 126. The borders of any cancerous regions of the resection sample 301 are calculated based on the spatial locations and corresponding classifications. For example, all of the spatial locations classified as “cancerous” may be analysed and those spatial locations, which are closest to an edge of the resection sample 301, may be determined. An outer perimeter may then be determined by fitting a contour line of coordinates across a 2-dimensional space (i.e. a 2D curve or spline) that follow the to the outermost “cancerous spatial locations”. The list of coordinates may be represented in a list of tuple format [(x1, y1), (x2, y2), (x3, y3) . . . ].

Optionally, the outer perimeter may be adjusted by moving it further away from the edge of the resected sample to ensure the capture of all diseased cells. The outer perimeter may be adjusted so that it contacts a buffer or margin zone of a specific micron width that is situated along the edge of the initial outer perimeter.

Based on the location of the outer perimeter or contour of the cancerous region, the contour circuitry 126 will determine if resection is complete or incomplete.

This may be determined based upon the detection of any part of the outer perimeter of the cancerous region to be within a threshold distance from the edge of the resection sample 301. Any such detected portions of the outer perimeter are determined to be a “positive margin” and resection is determined to be incomplete. If no part of the outer perimeter of the cancerous region is detected to be within a threshold distance from the edge of the resection sample 301, the resection is determined to be complete. The threshold distance may be, for example, 0.5, 1, or 2 mm.

Alternatively, the contour circuitry 126 may comprise a classification algorithm which, based on the spatial locations, the corresponding classifications, and spatial data indicating the edge of the tumour sample, assigns a probability that resection is incomplete. If the probability that resection is incomplete is over a threshold probability, the resection is determined to be incomplete. Otherwise, the resection is determined to be complete. The classification algorithm may be a machine learning algorithm comprising neural networks. The classification algorithm may be trained upon ground truth data comprising spatial locations and corresponding classifications obtained based upon an immunohistochemistry analysis of various tissue samples, spatial data indicating the edges of the respective various tumour samples, and a determination, by a clinician, if the resection of the respective various tumour samples is incomplete or complete. If resection is determined to be incomplete, the adjacent tissue sample 302 is sent for sectioning and profiling/sequencing.

In some embodiments, if resection is determined to be incomplete, an alert may be displayed on display screen 116. The alert may comprise an image of the resection sample 301, which shows the positive margin. The alert may further indicate the regions of the resection sample 301, which are determined to be healthy and the regions of the resection sample, which are determined to be cancerous. The alert may further indicate regions of the resection sample 301, which are suitable for dissection.

At 325, one or more subsections are dissected from the tissue sample 302 corresponding to regions of cancerous tissue, which are beyond the positive margin and located towards the edge of the tissue sample. In one embodiment, the subsections are dissected using microdissection, which is a method to isolate homogenous cell populations from specific microscopic regions of the tissue sample. In one embodiment, the subsections are dissected using laser capture microdissection

The genetic material in the dissected sections is then extracted and prepared for next-generation sequencing (NGS) using sample preparation protocols as known in the art (e.g. extraction of DNA from cells of the dissection sections, amplification of DNA by PCR, and library preparation for sequencing). NGS may be performed on the prepared genetic material to obtain genomic sequencing data, e.g. a plurality of reads, which are then aligned to a reference genome. Alignment may be performed using alignment tools as known in the art such as SAMtools or BCFtools.

At 330, the genetic profiling/sequencing data is received by the mutational profiling circuitry 128. The mutational profiling circuitry 128 determines genetic differences between the dissected resection sample at or near the positive margin and “healthy” tissue. The mutational profile circuitry may look for differences between the profiling/sequencing data of the dissected resection sample and profiling/sequencing data obtained from “healthy” tissue. The “healthy” tissue may be a biopsy of healthy tissue from the patient. As an alternative to determining the differences between the dissection resection sample at or near the positive margin and healthy tissue, the mutational profiling circuitry 128 may use bioinformatics approaches as known in the art (for example, the genomics analysis toolkit by the Broad Institute) to call variants in the profiling/sequencing data and subsequently identify those that are implicated in cancer. Such variants may be at locations of the genome corresponding to oncogenes or tumour suppressor genes. The implicated variants may be identified with reference to a catalogue of somatic mutations, such as the COSMIC database.

The output from this step is a set of mutations for the tumour, i.e. a tumour mutational profile, which is likely to correspond to the unresected tumour since the mutational profile is obtained based upon resected tissue located very close to tumour remaining in the patient.

At 335, the tumour mutational profile is optionally received by the CDS circuitry 130. Clinical decisions, such as the type of adjuvant therapy to be administer to the patient, the time to the next and subsequent scans, and the targets for a liquid biopsy, may be determined based on the number and/or types of mutants contained in the tumour mutational profile. This is described further with reference to FIG. 6.

With reference to FIGS. 4 and 5, methods to analyse spatial transcriptomics data to determine spatial locations as corresponding to healthy or cancerous tissue will now be described.

FIG. 4 shows a classification method 400 according to a gene set enrichment analysis approach will now be described. Gene set enrichment analysis (GSEA) is an approach to identify whether certain sets of genes are over- or under-represented in gene expression data.

At 405, the spatial transcriptomics data 340 is received by the classification circuitry 124.

At 410, the expression values for the plurality of spatial locations to be evaluated are normalized and the corresponding genes are then ranked. The expression values for the plurality of spatial locations are normalized based upon a normalization approach suitable for RNA sequencing data to provide normalized expression values in units of CPM (counts per million) RRKM (reads per kilobase million), or TPM (transcripts per kilobase million).

The genes are ranked based upon the normalized expression values and reference normalized single cell expression values. The reference normalized single cell expression values may be derived from healthy, or what is mostly healthy tissue. For example, the reference expression values may be derived from single cell gene expression data obtained from a biopsy of healthy tissue from the patient. Alternatively, the reference normalized single cell expression values may be an average of normalized single cell or bulk gene expression values from healthy tissue samples from other subjects. The normalized single cell gene expression values for healthy tissue samples from other subjects may be obtained from a genomics/transcriptomics data repository (for example the Genotype Tissue Expression Catalogue or the Human Cell Atlas).

The ranking may be performed according to a z-score calculated for each of the normalised expression values (i.e CPM normalized expression values, or normalized expression values according to some other chosen method), x. The z-score is calculated according to z=(x−μ)/σ, where μ is the mean and σ is the standard deviation of the corresponding reference normalized gene expression value. The z-score may be an absolute z-score (i.e. with a value between 0 and 1). The genes for each spatial location may then be ranked, from highest to lowest, according to the associated z-score value. As an alternative to ranking the genes based on z-score values, the genes may be ranked based on a differential expression levels calculated with reference to reference expression levels in healthy tissue.

At 415, GSEA is performed on the ranked list of genes using one or more predefined gene sets. GSEA may be performed using the GSEA software (UC San Diego and the Broad Institute). The GSEA algorithm uses statistical approaches to calculate an enrichment score for each predefined gene set. The gene sets may be oncogenic gene sets and they may correspond to the particular cancer to which the tissue sample relates to. The gene sets may be obtained from online databases such as MSigDB. The enrichment score corresponds to the extent that the genes in a predefined gene set feature at the top or bottom of the list of ranked genes. The statistical significance of the enrichment score is then estimated by comparing the enrichment score to a null distribution, which results in a p-value. The p-value may then be adjusted based on the size and number of the remaining pre-defined gene sets so the p-values can be compared.

GSEA is performed for each of the spatial locations to be evaluated. This results in one or more p-values being generated for anevaluated spatial location.

At 420, the one or more cells, which correspond to the spatial locations to be evaluated, are classified as healthy or cancerous based on the associated p-values. The one or more cells may be classified as cancerous if at least one of the p-values are lower than a threshold α, i.e. less than 0.05.

At 425, the plurality of spatial locations, and the corresponding classifications, are output.

With reference to FIG. 5, a machine-learning based classification method 500 will now be described. This method uses a machine-learning algorithm, which takes spatial transcriptomics data as input and predicts, based on the coordinates of the plurality of spatial locations and the gene expression levels associated with the plurality of spatial locations, whether the one or more cells corresponding to the plurality of spatial locations are cancerous or non-cancerous. The architecture of the machine-learning algorithm may comprise a plurality of neural network layers, and may comprise convolutional neural networks, transformers, and/or auto-encoders. The machine-learning algorithm may be an image segmentation algorithm, which processes the spatial transcriptomics data as if it were an image, wherein the spatial locations are equivalent to the locations of pixel points of an image, and the intensity of those pixels is the associated gene expression data.

The machine-learning algorithm may be trained based on ground truth datasets comprising spatial transcriptomics data obtained from resection samples and classifications of the plurality of spatial locations of the resection samples as cancerous or healthy. The ground truth classification may be based upon immunohistochemistry analysis.

At 505, the spatial transcriptomics data 340 is received by the contour circuitry 126.

At 510, the spatial transcriptomics data 340 is input to the machine-learning algorithm.

At 515, the plurality of spatial locations, and the corresponding classifications, are output.

With reference to FIG. 6, a method 600 to determine clinical decisions based on the mutational profile data will now be described.

At 605, the tumour mutational profile data is received by the CDS circuitry 130. Subsequent to this, any one or more of 610, 615 and 620 may be performed.

At 610, the time to the next and subsequent scans, such as PET or MRI scans, may be determined based on the types and numbers of mutants contained in the tumour mutational profile data. For example, if there are a large number of mutants, or one or more mutants associated with an aggressive form of cancer, the next scan may be scheduled to be relatively sooner, and subsequent scans may be scheduled to occur at a high frequency. If there are low number of mutants and no mutants associated with an aggressive form of cancer, a/the subsequent scan(s) may be scheduled to occur at a lower and/or more distant frequency.

At 615, a type of adjuvant therapy, such as chemotherapy, radiation therapy, hormone therapy, or biological therapy, may be determined based on the tumour mutational data. Approved guidelines, such as the NICE guidelines, may be used to provide a pharmacogenetic recommendation or therapy suggestion based upon the tumour mutational data. For example, using the NICE guidelines, osimertinib may be recommended for lung cancer based upon presence of a EGFR T790M mutation when the lung cancer is locally advanced or metastatic. Optionally, further surgery may be recommended based on the tumour mutational data.

At 620, the targets of a liquid biopsy may be determined based on the tumour mutational profile. Liquid biopsies can be used to detect the presence of specific biomarkers, such as genetic mutations, in the blood. The targets of the liquid biopsy may be chosen to correspond to mutants in the mutational profile data such that the liquid biopsy can specifically monitor the progression of the unresected tumour.

It is envisaged that in further embodiments, a further type of omics analysis may be performed based on a further adjacent tissue sample to aid in the detection of the positive margin and/or to characterise the tumour. The further type of omics analysis may be, for example, proteomics or metabolomics. A workflow for performing proteomics and metabolomics on a tissue sample is described in Gegner, Hagen M., et al. “A single-sample workflow for joint metabolomic and proteomic analysis of clinical specimens.” Clinical Proteomics 21.1 (2024): 49.

Advantageously, the embodiments described herein provide a way to identify a positive margin of a resection sample using spatial transcriptomics. By using high-throughput data to analyse a resection sample, a positive margin may be identified with greater accuracy than what can be achieved using immunohistochemistry methods. Furthermore, the embodiments described herein provide a way to extract mutational data specific to regions at or adjacent to a positive margin. Since the positive margin is close to any unresected tumour within the patient, the mutational profile data allows for the remaining tumour to be characterised. This allows for a more targeted understanding of the genomic make-up of the cancer tissue remaining after incomplete resection. This further allows for a precision-medicine approach to be employed when determining the best treatment for a patient after incomplete resection. For example, a treatment type which is best suited to the types, and number, of mutations on the tumour can be employed which can lead to better treatment outcomes and may reduce the overall cost in treating a patient.

As well as guiding clinical decisions, the spatial transcriptomics data and sequencing data as described herein can be used to aid clinical research. For example, the data can be used to understand the spatial and clonal structure of tumours, and can be used to understand the properties of tumours, which are difficult to resect.

Whilst particular circuitries have been described herein, in alternative embodiments, functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

A method for detecting incomplete tumour resection and informing precision adjuvant therapy using spatially directed residual disease characterisation where
1. Resected tissue is sectioned and spatial transcriptomics performed (herein referred to as the Atlas Section)
2. The borderzone of the resected tissue in the Atlas Section is assessed for gene expression signatures
3. If the borderzone in the Atlas Section indicates diseased cells at any axis of resection made
   1. An adjacent section is laser capture microdissected at these coordinates (herein referred to as the Genomics Section)
   2. These laser captured cells are characterised genomically
   3. The findings of this genomic characterisation are used to inform choice of adjuvant intervention.

Optionally, the intended clinical decision based on characterisation is not adjuvant intervention but another clinical decision (ie, time to next surgery/scan).

Optionally, the Atlas Section provides relevant coordinates to another section, the purpose of which is to conduct alternative omics assessment than Genomics.

Optionally, an alternate form of microdissection to laser-capture is performed on the Genomics Section

Optionally, the user may wish to define a buffer/margin zone of specific micron width along the edge of the contour of the Altas section in order to ensure capture of all diseased cells

According to a first aspect there is provided a method for analysing a tissue sample, the method comprising: receiving spatial transcriptomics data based upon the tissue sample, the spatial transcriptomics data comprising, for a plurality of spatial locations on the sample, expression values of a plurality of genomic nucleic acids associated with one or more cells located at the plurality of spatial locations; for at least a subset of the spatial locations, classifying the corresponding one or more cells as cancerous or non-cancerous based upon the associated expression values; and determining a boundary of a cancerous region of the tissue sample based upon the spatial locations classified as corresponding to one or more cancerous cells.

Optionally, the tissue sample is a resection surgery tissue sample, and the method further comprises determining the resection as incomplete or complete based upon the position of the boundary of the cancerous region.

Optionally, the method further comprises providing an alert in response to determining the resection as incomplete.

Optionally, resection is determined as incomplete if a boundary of a cancerous region is located within a threshold distance from an edge of the resection sample.

Optionally, the tissue sample is a first tissue sample, and the method further comprises: obtaining a second tissue sample associated with the first tissue sample, obtaining one or more subsections of the second tissue sample corresponding to cancerous tissue located at, or adjacent to, the edge of the first tissue sample, genetic profiling the one or more subsections of the second tissue sample to obtain genetic information; and characterising a mutational profile of the cancerous region based on the genetic information.

Optionally, the first tissue sample and the second tissue sample are adjacent slices from a larger tissue sample.

Optionally, the one or more subsections are obtained by laser capture microdissection.

Optionally, classification of one or more cells corresponding to the at least a subset of the plurality of spatial locations as cancerous or non-cancerous is based upon the associated expression values and reference expression values for one or more predetermined gene sets.

Optionally, the classification of one or more cells corresponding to the at least a subset of the plurality of spatial locations is performed by: ranking the genes associated with the least a subset of the plurality of spatial locations based upon a comparison of the associated expression values with reference gene expression values, running a gene set enrichment analysis algorithm on the ranked genes using the predetermined gene sets to produce one or more p-values, and classifying the one or more cells as cancerous if one or more of the p-values are less than a threshold.

Optionally, classification of one or more cells corresponding to the at least a subset of the plurality of spatial locations as cancerous or non-cancerous is based upon a machine learning algorithm trained to classify one or more cells as cancerous or non-cancerous based upon the associated expression values and associated spatial location.

Optionally, the method is used in a clinical setting to facilitate further clinical decisions following resection surgery.

Optionally, the method further comprises: determining a clinical decision based on the mutational profile data, and/or providing the mutational profile data to a clinician to facilitate them in making a clinical decision.

Optionally, the clinical decision is a time to next surgery and/or a time to a next scan, and/or type of therapy such as chemotherapy.

Optionally, the tissue sample is a first tissue sample and the method further comprises: obtaining a third tissue sample associated with the first tissue sample, obtaining one or more subsections of the third tissue sample corresponding to cancerous tissue located at, or adjacent to, the edge of the first tissue sample, performing proteomics and/or metabolomics of the one or more subsections of the second tissue sample to obtain proteomic and/or metabolomic information; and characterising the cancerous region based on the proteomic and/or metabolomic information.

Optionally, the genomic nucleic acids are genes.

According to a second aspect there is provided medical information processing apparatus comprising a processing circuitry configured to: receive spatial transcriptomics data based upon the tissue sample, the spatial transcriptomics data comprising, for a plurality of spatial locations on the sample, expression values of a plurality of genomic nucleic acids corresponding to one or more cells located at the plurality of spatial locations; for at least a subset of the plurality of spatial locations, classify the corresponding one or more cells as cancerous or non-cancerous based upon the associated expression values; and determine a boundary of a cancerous region of the tissue sample based upon the spatial locations classified as corresponding to one or more cancerous cells.

According to a third aspect there is provided a non-transitory storage medium storing therein a program that causes a computer to execute the steps of:: receive spatial transcriptomics data based upon the tissue sample, the spatial transcriptomics data comprising, for a plurality of spatial locations on the sample, expression values of a plurality of genomic nucleic acids associated with one or more cells located at the plurality of spatial locations; for at least a subset of the plurality of spatial locations, classify the corresponding one or more cells as cancerous or non-cancerous based upon the associated expression values; and determine a boundary of a cancerous region of the tissue sample based upon the spatial locations classified as corresponding to one or more cancerous cells.

1. A method for analysing a tissue sample, the method comprising:
receiving spatial transcriptomics data based upon the tissue sample, the spatial transcriptomics data comprising, for a plurality of spatial locations on the sample, expression values of a plurality of genomic nucleic acids associated with one or more cells located at the plurality of spatial locations;
for at least a subset of the plurality of spatial locations, classifying the corresponding one or more cells as cancerous or non-cancerous based upon the associated expression values; and
determining a boundary of a cancerous region of the tissue sample based upon the spatial locations classified as corresponding to one or more cancerous cells.

2. The method according to claim 1, wherein the tissue sample is a resection surgery tissue sample, the method further comprising:
determining the resection as incomplete or complete based upon the position of the boundary of the cancerous region.

3. The method according to claim 2, further comprising:
providing an alert in response to determining the resection as incomplete.

4. The method according to claim 2, wherein resection is determined as incomplete if a boundary of a cancerous region is located within a threshold distance from an edge of the resection sample.

5. The method according to claim 1, wherein the tissue sample is a first tissue sample, the method further comprising:
obtaining a second tissue sample associated with the first tissue sample,
obtaining one or more subsections of the second tissue sample corresponding to cancerous tissue located at, or adjacent to, the edge of the first tissue sample,
genetic profiling the one or more subsections of the second tissue sample to obtain genetic information; and
characterising a mutational profile of the cancerous region based on the genetic information.

6. The method according to claim 5, wherein the first tissue sample and the second tissue sample are adjacent slices from a larger tissue sample.

7. The method according to claim 5, wherein the one or more subsections are obtained by laser capture microdissection.

8. The method according to claim 1, wherein classification of one or more cells corresponding to theat least a subset of the plurality of spatial locations as cancerous or non-cancerous is based upon the associated expression values and reference expression values for one or more predetermined gene sets.

9. The method according to claim 8, wherein the classification of one or more cells corresponding to the at least a subset of the plurality of spatial locations is performed by:
ranking the genes associated with the at least a subset of the plurality of spatial locations based upon a comparison of the associated expression values with reference gene expression values,
running a gene set enrichment analysis algorithm on the ranked genes using the predetermined gene sets to produce one or more p-values, and
classifying the one or more cells as cancerous if one or more of the p-values are less than a threshold.

10. The method according to claim 1, wherein classification of one or more cells corresponding to the at least a subset of the plurality of spatial locations as cancerous or non-cancerous is based upon a machine learning algorithm trained to classify one or more cells as cancerous or non-cancerous based upon the associated expression values and associated spatial location.

11. The method according to claim 1, method being used in a clinical setting to facilitate further clinical decisions following resection surgery.

12. The method according to claim 11 when dependent on claim 5, further comprising:
determining a clinical decision based on the mutational profile data, and/or
providing the mutational profile data to a clinician to facilitate them in making a clinical decision.

13. The method according to claim 12, wherein the clinical decision is a time to next surgery and/or a time to a next scan, and/or type of therapy such as chemotherapy.

14. The method according to claim 1, wherein the tissue sample is a first tissue sample, the method further comprising:
obtaining a third tissue sample associated with the first tissue sample,
obtaining one or more subsections of the third tissue sample corresponding to cancerous tissue located at, or adjacent to, the edge of the first tissue sample,
performing proteomics and/or metabolomics of the one or more subsections of the second tissue sample to obtain proteomic and/or metabolomic information; and
characterising the cancerous region based on the proteomic and/or metabolomic information.

15. The method according to claim 1, wherein the genomic nucleic acids are genes.

16. A medical information processing apparatus comprising a processing circuitry configured to:
receive spatial transcriptomics data based upon the tissue sample, the spatial transcriptomics data comprising, for a plurality of spatial locations on the sample, expression values of a plurality of genomic nucleic acids corresponding to one or more cells located at the plurality of spatial locations;
for at least a subset of the spatial locations, classify the corresponding one or more cells as cancerous or non-cancerous based upon the associated expression values; and
determine a boundary of a cancerous region of the tissue sample based upon the spatial locations classified as corresponding to one or more cancerous cells.

17. A non-transitory storage medium storing therein a program that causes a computer to execute the steps of:
receive spatial transcriptomics data based upon the tissue sample, the spatial transcriptomics data comprising, a plurality of spatial locations on the sample, expression values of a plurality of genomic nucleic acids associated with one or more cells located at the plurality of spatial locations;
for at least a subset of the spatial locations, classify the corresponding one or more cells as cancerous or non-cancerous based upon the associated expression values; and
determine a boundary of a cancerous region of the tissue sample based upon the spatial locations classified as corresponding to one or more cancerous cells.
